(19) United States
(12) Patent Application Publication
(10) Pub. No.: US 2022/0218537 A1
(43) Pub. Date: Jul. 14, 2022

(54) ABSORBENT ARTICLE

(71) Applicant: Kimberly-Clark Worldwide, Inc., Neenah, WI (US)

(72) Inventors: JuHyung Lee, YoungTong-gu, Suwon-si (KR); SungSu Kim, Suji-gu, Yongin-si (KR); EunSoo Cho, Suji-gu, Yongin-si (KR); SeoYeon Son, Bundang-gu, Seongnam-si (KR); SooYong Cho, Bundang-gu, Seongnam-si (KR); KueYoung You, Sujeong-gu, Seongnam-si (KR)

(21) Appl. No.: 17/595,338

(22) PCT Filed: May 15, 2019
(86) PCT No.: PCT/US19/32372

§ 371 Date: Nov. 15, 2021

(51) Int. Cl. A61F 13/512; A61F 13/513; A61F 13/514; A61F 13/534

(52) U.S. Cl. CPC A61F 13/512; A61F 13/534; A61F 13/51496; A61F 13/51394

(57) ABSTRACT

An absorbent article can have a topsheet layer, a backsheet layer, and an absorbent core positioned between the topsheet layer and the backsheet layer. The topsheet layer can be formed of a topsheet material having an opacity of at least 40%. The backsheet layer can be formed of a backsheet material having an opacity less than 30%. A portion of the area of the topsheet layer is defined by openings within the topsheet layer. The opacity of the topsheet layer can make tire openings visible to the wearer of the absorbent article and identify to the wearer that the absorbent article lias the desired attribute of breathability. Additionally, the openings can have a minimum size such that the color of the wearer's undergarment can be visible through the openings and through the backsheet layer thereby providing tire wearer with an absorbent article having the desired attribute of discretion.

### BACKGROUND OF THE DISCLOSURE

Products such as absorbent articles are often used to collect and retain human body exudates containing, for example, urine, menses and/or blood. Comfort, absorbency, and discretion are three main product attributes and areas of concern for the wearer of the product. In particular, a wearer is often interested in knowing that such products will absorb significant volumes of body exudates with minimal leakage in order to protect their undergarments, outer garments, or bedsheets from staining, and that such products will help them avoid the subsequent embarrassment brought on by such staining.

Currently, a wide variety of products for absorption of body exudates are available in the form of feminine pads, sanitary napkins, panty shields, pantiliners, and incontinence devices. These products generally have an absorbent core positioned between a body-facing liquid permeable topsheet layer and a garment-facing liquid impermeable backsheet layer. The edges of the topsheet and the backsheet layers are often bonded together at their periphery to form a seal to contain the absorbent core and body exudates received into the product through the topsheet layer. In use, such products are typically positioned in the crotch portion of an undergarment for absorption of the body exudates and a garment attachment adhesive on the backsheet layer can be used to attach the product to the inner crotch portion of the undergarment.

Due to changing trends within the fashion industry towards more form-fitting and/or translucent clothing material, wearers of such conventional absorbent products have a desire for the absorbent product to be discreet when worn. Such discretion is desirable with regard to the color of the absorbent product such that the absorbent product is not visible through a wearer's clothing. In order to meet this consumer desire, manufacturers have designed products to incorporate a variety of colors to match the color of clothing material. However, the number of colors available to consumers for their clothing options is very large and manufacturers are not able to keep up with the large number of color options. Manufacturers have, therefore, started to design absorbent products wherein the topsheet layer and the backsheet layer, and in some cases, the absorbent core, are transparent such that the color of the wearer's undergarment is visible through the layers of the absorbent product and the absorbent product can, therefore, blend in with the wearer's undergarment.

Some drawbacks, however, exist with such absorbent product designs. In addition to having a discreet absorbent product, wearers of such conventional absorbent products are interested in having such products demonstrate that they are breathable during usage (for both skin health rationale as well as physical comfort) and demonstrate the ability to contain body exudate without leakage of such body exudate from the absorbent article. Manufacturers have attempted to design a product which is breathable such as by utilizing a nonwoven material as the backsheet layer, however, such materials have not protected against leakage of body exudates.

Therefore the problem exists as to how to provide an absorbent product which is discreet and which provides the wearer with the confidence that the product will be breathable and will protect them against leakage of body exudates from the product. There is a need to provide an improved absorbent product, such as an absorbent article, which can provide the desired attributes of discretion, breathability, and protection from body exudate leakage to the wearer of the absorbent article.

### SUMMARY OF THE DISCLOSURE

In various embodiments, an absorbent article can have a perimeter defined by a first transverse direction end edge and a second transverse direction end edge opposed to the first transverse direction end edge and an opposing pair of longitudinal direction side edges extending between and connecting the first transverse direction end edge and the second transverse direction end edge; a topsheet layer which can have a topsheet material having an opacity of at least 40%, a body facing surface, and an absorbent core facing surface; an area defined by the perimeter of the absorbent article; a plurality of openings extending from the body facing surface of the topsheet material to the absorbent core facing surface of the topsheet material wherein each opening provides an open space within the area of the topsheet layer; a backsheet layer comprising a backsheet material having an opacity less than 30%; and an absorbent core positioned between the topsheet layer and the backsheet layer.

In various embodiments, the opacity of the topsheet material is at least 45%. In various embodiments, the opacity of the backsheet material is less than 25%. In various embodiments, an opacity of the absorbent core is at least the same as the opacity of the topsheet material. In various embodiments, an opacity of the absorbent core is greater than the opacity of the topsheet material.

In various embodiments, a size dimension of the absorbent core is from 15% to 60% of a size dimension of the absorbent article.

In various embodiments, the area of the topsheet layer contains from 15% to 40% of open space. In various embodiments, each opening of the plurality of openings has a size dimension greater than 0.3 mm². In various embodiments, each opening of the plurality of openings has a size dimension greater than 0.7 mm².

In various embodiments, the absorbent article can further have a secondary absorbent layer. In various embodiments, the secondary absorbent layer is positioned between the absorbent core and the backsheet layer. In various embodiments, the secondary absorbent layer has an opacity less than 30%. In various embodiments, the secondary absorbent layer has an opacity less than 25%.

### BRIEF DESCRIPTION OF DRAWINGS

FIG. 1 is a top down view of an embodiment of an absorbent article.

FIG. 2 is an exploded side view of an embodiment of the absorbent article of FIG. 1.

FIG. 3 is an exploded end view of the absorbent article of FIG. 2.

FIG. 4 is an exploded side view of another embodiment of the absorbent article of FIG. 1.

FIG. 5 is an exploded end view of the absorbent article of FIG. 4.

FIG. 6 is a top down view of another embodiment of an absorbent article.

FIG. 7 is an exploded side view of an embodiment of the absorbent article of FIG. 6.

FIG. 8 is an exploded side view of another embodiment of the absorbent article of FIG. 6.

Repeat use of reference characters in the present specification and drawings is intended to represent the same or analogous features or elements of the disclosure.

### DETAILED DESCRIPTION OF THE DISLOSURE

The present disclosure is generally directed towards an absorbent article which can provide discretion and breathability. The absorbent article can have a topsheet layer, a backsheet layer, and an absorbent core positioned between the topsheet layer and the backsheet layer. The topsheet layer can be formed of a topsheet material having an opacity of at least 40%. The backsheet layer can be formed of a backsheet material having an opacity less than 30%. A portion of the area of the topsheet layer is defined by openings within the topsheet layer. The opacity of the topsheet layer can make the openings in the topsheet layer visible to the wearer of the absorbent article identifying to the wearer of the absorbent article that the absorbent article has the desired attribute of breathability. Additionally, the openings within the topsheet layer can have a minimum size such that the color of the wearer's undergarment can be visible through the openings of the topsheet layer and through the backsheet layer thereby providing the wearer with an absorbent article having the desired attribute of discretion.

Definitions:

As used herein, the term “absorbent article” refers herein to a garment or other end-use personal care absorbent article, including, but not limited to, catamenial products, such as sanitary napkins, feminine pads, pantiliners, and panty shields, incontinence devices, and the like.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form one fiber.

Conjugate fibers are also sometimes referred to as bicomponent fibers or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al. each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “hydrophilic” refers to surfaces with a water contact angle at or below 59°.

As used herein, the term “hydrophobic” refers to surfaces with the property to repel fluid with a water contact angle at or greater than 90°.

As used herein, the term “semi-hydrophilic” refers to surfaces with a water contact angle from 60° to 89°.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Patent No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10 or 20 gsm to about 120, 125 or 150 gsm.

As used herein, the term “opacity” refers herein to the ability of a material to block the transmission of visible light through the body of the material. Opacity can be measured using a standard colorimeter. The closer an opacity measurement is to 100%, the more opaque the material is.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Patent No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in part on ionicity (a factor of the ionic concentration of the aqueous solution), and the SAP functional polar groups that have an affinity for water. SAP are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating on another material or fiber.

Absorbent Article:

The present disclosure is generally directed towards an absorbent article which can provide discretion and breathability. The absorbent article can have a topsheet layer, a backsheet layer, and an absorbent core positioned between the topsheet layer and the backsheet layer. The topsheet layer can be formed of a topsheet material having an opacity of at least 40%. The backsheet layer can be formed of a backsheet material having an opacity less than 30%. A portion of the area of the topsheet layer is defined by openings within the topsheet layer. The opacity of the topsheet layer can make the openings in the topsheet layer visible to the wearer of the absorbent article identifying to the wearer of the absorbent article that the absorbent article has the desired attribute of breathability. Additionally, the openings within the topsheet layer can have a minimum size such that the color of the wearer's undergarment can be visible through the openings of the topsheet layer and through the backsheet layer thereby providing the wearer with an absorbent article having the desired attribute of discretion.

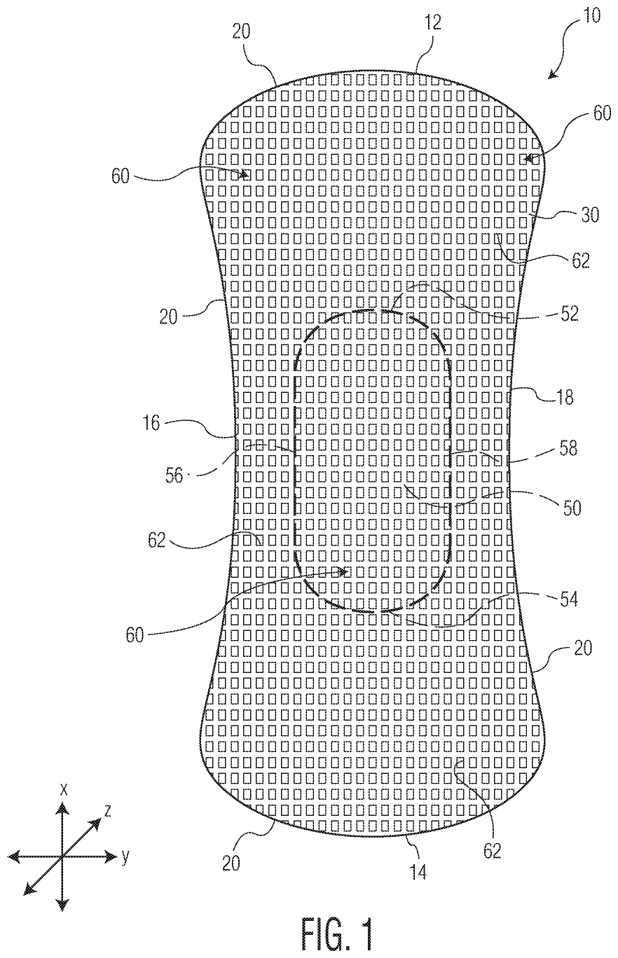

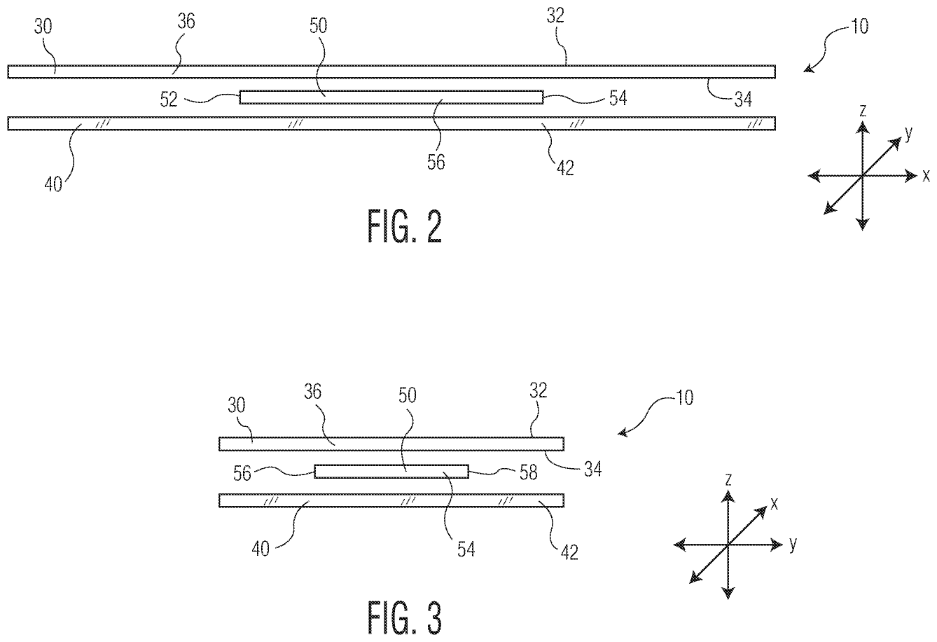

Referring to FIGS. 1-3, FIG. 1 provides an illustration of a top down view of an exemplary absorbent article 10, FIG. 2 provides an illustration of an exploded side view of the absorbent article 10 of FIG. 1, and FIG. 3 provides an illustration of an exploded end view of the absorbent article 10 of FIG. 2. The absorbent article 10 can have a longitudinal direction (X), a transverse direction (Y), and a depth direction (Z). The absorbent article 10 can have a first transverse direction end edge 12, a second transverse direction end edge 14 opposite the first transverse direction end edge 12, and a pair of opposing longitudinal direction side edges, 16 and 18, extending between and connecting the first transverse direction end edge 12 and the second transverse direction end edge 14. In various embodiments, the absorbent article 10 can take on various geometries but will generally have a pair of opposing transverse direction end edges, 12 and 14, and a pair of opposing longitudinal direction side edges, 16 and 18. Each of the edges, 12, 14, 16, and 18, form the overall perimeter 20 of the absorbent article 10 defining the overall shape of the absorbent article 10. The absorbent article 10 can have a wearer facing, liquid permeable topsheet layer 30 and a garment facing, liquid impermeable backsheet layer 40. An absorbent core 50 can be positioned between the topsheet layer 30 and the backsheet layer 40. The perimeter 20, in addition to defining the overall geometry of the absorbent article 10, can also define the overall shape of each of the topsheet layer 30 and backsheet layer 40.

The topsheet layer 30 and the backsheet layer 40 can both extend beyond the outermost peripheral edges of the absorbent core 50 and can be peripherally bonded together, either entirely or partially, using known bonding techniques to form a sealed peripheral region. For example, the topsheet layer 30 and the backsheet layer 40 can be bonded together by adhesive bonding, ultrasonic bonding, or any other suitable bonding method known in the art.

Topsheet Layer:

The topsheet layer 30 defines a body facing surface 32 of the absorbent article 10 that may directly contact the body of the wearer and is liquid permeable to receive body exudates. Opposite the body facing surface 32 of the topsheet layer 30 is the absorbent core facing surface 34 of the topsheet layer 30 which faces towards the absorbent core 50 of the absorbent article 10. The topsheet layer 30 is desirably provided for comfort and functions to direct body exudates away from the body of the wearer, through its own structure, and towards the absorbent core 50. The topsheet layer 30 desirably retains little to no liquid in its structure, so that it provides a relatively comfortable and non-irritating surface next to the skin of the wearer of the absorbent article 10.

The topsheet layer 30 can be formed from a topsheet material 36 wherein the topsheet material 36 can be a single layer of material, or alternatively, can be multiple layers that have been laminated together. The topsheet material 36 of the topsheet layer 30 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form the topsheet material 36 of the topsheet layer 30.

In various embodiments the topsheet material 36 of the topsheet layer 30 can be constructed from various nonwoven webs such as meltblown webs, spunbond webs, hydroentangled spunlace webs, or through air bonded carded webs. Examples of suitable materials for the topsheet material 36 for the topsheet layer 30 can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of topsheet material 36 suitable for a topsheet layer 30 is a natural fiber web formed from 100% cotton. An additional example of a suitable topsheet layer 30 is one formed from a topsheet material 36 which is a bonded carded web made of polypropylene and polyethylene such as that obtainable from Sandler Corp., Germany. U.S. Pat. No. 4,801,494 to Datta, et al., and U.S. Pat. No. 4,908,026 to Sukiennik, et al., and WO 2009/062998 to Texol teach various other topsheet materials 36 that may be used as the topsheet layer 30, each of which is hereby incorporated by reference thereto in its entirety. Additional topsheet materials 36 for a topsheet layer 40 can include, but are not limited to, those described in U.S. Patent No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 30 can contain a plurality of openings 60. Each opening 60 can extend from the body facing surface 32 of the topsheet layer 30 to the absorbent core facing surface 34 of the topsheet layer 30 to permit body exudates to pass more readily into the absorbent core 50. The openings 60 may be randomly or uniformly arranged throughout the topsheet layer 30. Each opening 60 can be in the shape of a circle, oval, square, rectangle, diamond, or any other geometric shape deemed suitable. Each opening 60 can be bounded by a perimeter 62 which defines the size of each opening 60 and, therefore, the amount of open space within the topsheet layer 30 due to the presence of the opening 60. The size of each opening 60 is at least 0.3, 0.5, 0.7, 1.0, 1.2, 1.4, 1.8, 2.0, 2.2, 2.4, 2.6, 2.8, 3.0, or 3.2 mm². The perimeter 20 of the absorbent article 10 can define the area of the topsheet layer 30. In various embodiments, the number of openings 60 present in the topsheet layer 30 in combination with the size of each opening 60 in the topsheet layer 30 can result in the area of the topsheet layer 30 containing from 15, 20, or 25% to 30, 35, or 40% of open space due to the presence of the plurality of openings 60.

In various embodiments, the topsheet material 36 forming the topsheet layer 30 can have an opacity of at least 40%. In various embodiments, the topsheet material 36 forming the topsheet layer 30 can have an opacity of at least 45%. The opacity of the topsheet material 36 forming the topsheet layer 30 is measured prior to the incorporation of any openings 60 into the topsheet layer 30. The opacity of the topsheet material 36 forming the topsheet layer 30 in combination with the size of the openings 60 can visually highlight the presence of the openings 60 within the topsheet layer 30.

In various embodiments, the topsheet layer 30 can have a basis weight ranging from about 5, 10, 15, 20, or 25 gsm to about 50, 100, 120, 125, or 150 gsm. For example, in an embodiment, a topsheet layer 30 can be constructed from a through air bonded carded web having a basis weight ranging from about 15 gsm to about 100 gsm. In another example, a topsheet layer 30 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics, and others. Alternatively, apertured films, such as those available from such film suppliers as Texol, Italy and Tredegar, U.S.A. may be utilized. In various embodiments, the topsheet layer 30 can be constructed from a perforated polyethylene film having a basis weight from about 15 gsm to about 30 gsm. In various embodiments, the topsheet layer 30 can be constructed from a cotton material and have a basis weight from about 25 gsm to about 35 gsm.

In various embodiments, the topsheet layer 30 can be at least partially hydrophilic. In various embodiments, a portion of the topsheet layer 30 can be hydrophilic and a portion of the topsheet layer 30 can be hydrophobic. In various embodiments, the portions of the topsheet layer 30 which can be hydrophobic can be either an inherently hydrophobic material or can be a material treated with a hydrophobic coating.

Absorbent Core:

An absorbent core 50 can be positioned between the topsheet layer 30 and the backsheet layer 40. The absorbent core 50 is designed to absorb body exudates, including menstrual fluid, blood, urine, and other bodily fluids such as sweat and vaginal discharge.

The absorbent core 50 can generally be any single layer structure or combination of layer components, which can demonstrate some level of compressibility, conformability, be non-irritating to a wearer's skin, and capable of absorbing and retaining liquids and other body exudates. In various embodiments, the absorbent core 50 can be formed from a variety of different materials and can contain any number of desired layers. In various embodiments in which the absorbent core 50 is a multi-layered structure, each of the layers can contain similar materials or different materials. For example, the absorbent core 50 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of a wood pulp fluff can be identified with the trade designation NB 416, available from Weyerhaeuser Corp., and is a bleached, highly absorbent wood pulp containing primarily soft wood fibers. By way of example, suitable materials and/or structures for the absorbent core 52 can include, but are not limited to, those described in U.S. Patent No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al., each of which is hereby incorporated by reference thereto in its entirety.

In various embodiments, if desired, the absorbent core 50 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 50 in any amount as desired.

Regardless of the combination of absorbent materials used in the absorbent core 50, the absorbent materials can be formed into a web structure by employing various conventional methods and techniques. For example, the absorbent web can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art.

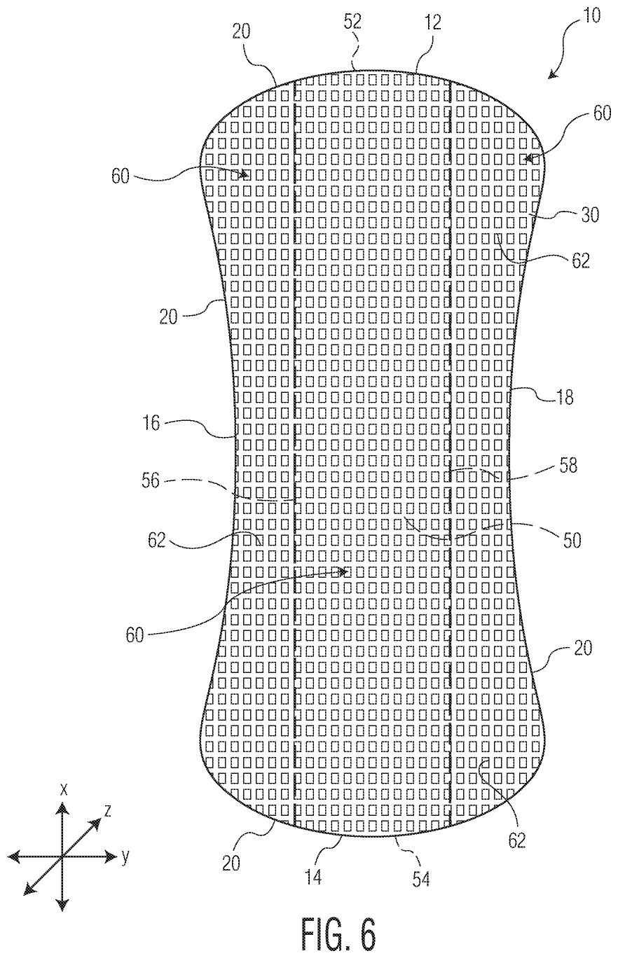

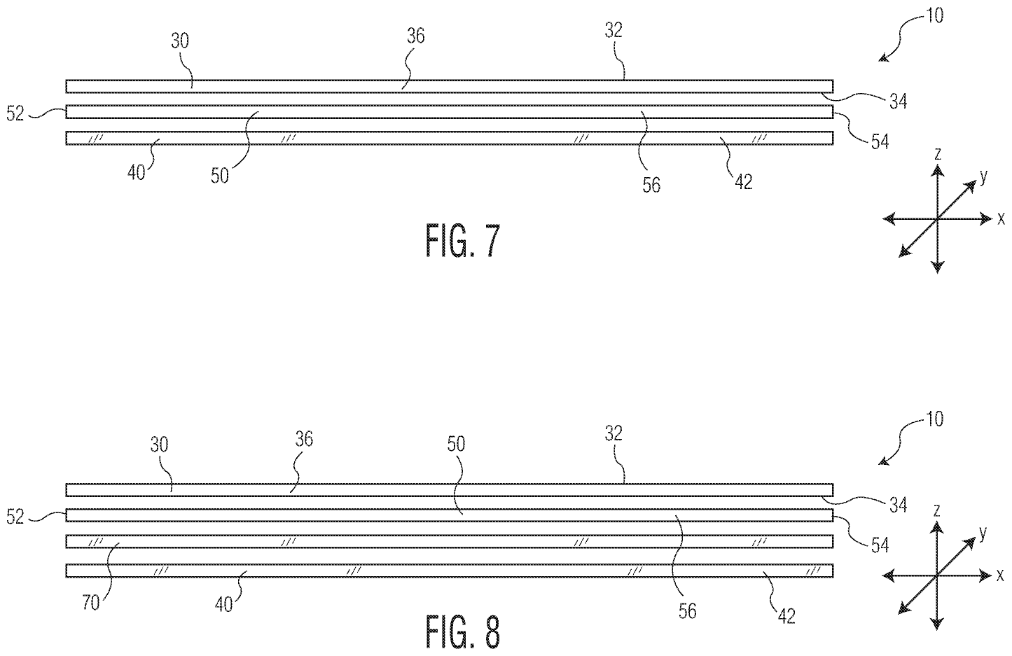

The absorbent core 50 can have a first transverse direction core end edge 52 as well as a second transverse direction core end edge 54 opposite the first transverse direction core end edge 52. The absorbent core 50 can also have a pair of opposing longitudinal direction core side edges, 56 and 58. The combination of each of the transverse direction core end edges, 52 and 54, and the longitudinal direction core side edges, 56 and 58, provide an overall shape and size dimension of the absorbent core 50. The absorbent core 50 can be provided in any shape as deemed suitable for the absorbent article 10 such as, but not limited to, circular, oblong, oval, rectangular, tear-dropped, hourglass, racetrack, triangular, dog-bone, and elliptical. In various embodiments, the shape of the absorbent core 50 can have a shape which provides symmetry about at least one axis, longitudinal and/or transverse, of the absorbent article 10. In various embodiments, the shape of the absorbent core 50 can be one in which there is no symmetry of the absorbent core 50 about either of the axes, longitudinal or transverse of the absorbent article 10. The size dimension of the absorbent core 50 can be less than the size dimension of the overall absorbent article 10, in order to be adequately contained therein. In various embodiments, the size dimension of the absorbent core 50 is from 15, 20, 25, or 30% to 35, 40, 45, 50, 55, or 60% of the size dimension of the absorbent article 10 wherein the size dimension of the absorbent article 10 is defined by the perimeter 20 of the absorbent article 10. In various embodiments, such as illustrated in FIGS. 1-5, the absorbent core 50 can be of a generally ovular shape and can be centered within the absorbent article 10. As illustrated in FIGS. 1-5, the absorbent core 50 can have a size dimension which is approximately 20% of the size dimension of the absorbent article 10. In various embodiments, such as illustrated in FIGS. 6-8, the absorbent core 50 can be an elongated rectangular shape and can extend from a portion of the first transverse direction edge 12 to a portion of the second transverse direction edge 14 of the absorbent article 10. As illustrated in FIGS. 6-8, the absorbent core 50 can have a size dimension which is approximately 50% of the size dimension of the absorbent article 10.

As described above, in various embodiments, an absorbent core 50 can be a single layer structure and can include, for example, a matrix of cellulosic fluff and superabsorbent material. In various embodiments, an absorbent core 50 can have at least two layers of material, such as, for example, a body facing layer and a garment facing layer. In various embodiments, the two layers can be identical to each other. In various embodiments, the two layers can be different from each other. In such embodiments, the two layers can provide the absorbent article 10 with different absorption properties as deemed suitable. In various embodiments, the body facing layer of the absorbent core 50 may be constructed of through-air bonded carded web material and the garment facing layer of the absorbent core 50 may be constructed of a compressed sheet of cellulosic pulp. In such embodiments, the through-air bonded carded web material can have a basis weight from about 20 to about 30 gsm and the compressed sheet of cellulosic pulp can have a basis weight from about 80 to about 120 gsm.

In various embodiments, the absorbent core 50 can have an opacity which is the same as the opacity of the topsheet material 36 forming the topsheet layer 30. In various embodiments, the absorbent core 50 can have an opacity which is greater than the opacity of the topsheet material 36 forming the topsheet layer 30. In various embodiments, the absorbent core 50 can have an opacity of at least 40%. In various embodiments, the absorbent core 50 can have an opacity of at least 45%.

Secondary Absorbent Layer:

In various embodiments, an absorbent article 10 can have an additional layer in the absorbent article 10 which can be a secondary absorbent layer 70. A secondary absorbent layer 70 can be constructed of any woven or nonwoven material that is easily penetrated by body exudates. The secondary absorbent layer 70 can help to absorb, decelerate, and diffuse surges or gushes of liquid that may be rapidly introduced into the absorbent article 10. Various woven fabrics and nonwoven webs can be used to construct the secondary absorbent layer 70. For example, the secondary absorbent layer 70 can comprise a nonwoven fabric layer composed of a meltblown or spunbond web of polyolefin or polyester filaments. Such nonwoven fabric layers may include conjugate, biconstituent and homopolymer fibers of staple or other lengths and mixtures of such fibers with other types of fibers. The secondary absorbent layer 70 can also be a bonded card web or an airlaid web composed of natural and/or synthetic fibers. The bonded carded web may, for example, be a powder bonded carded web, an infrared bonded carded web, or a through air bonded carded web. The bonded carded webs can optionally include a mixture or blend of different fibers. The secondary absorbent layer 70 typically has a basis weight of less than about 100 gsm, and in some embodiments, from about 10 gsm to about 40 gsm.

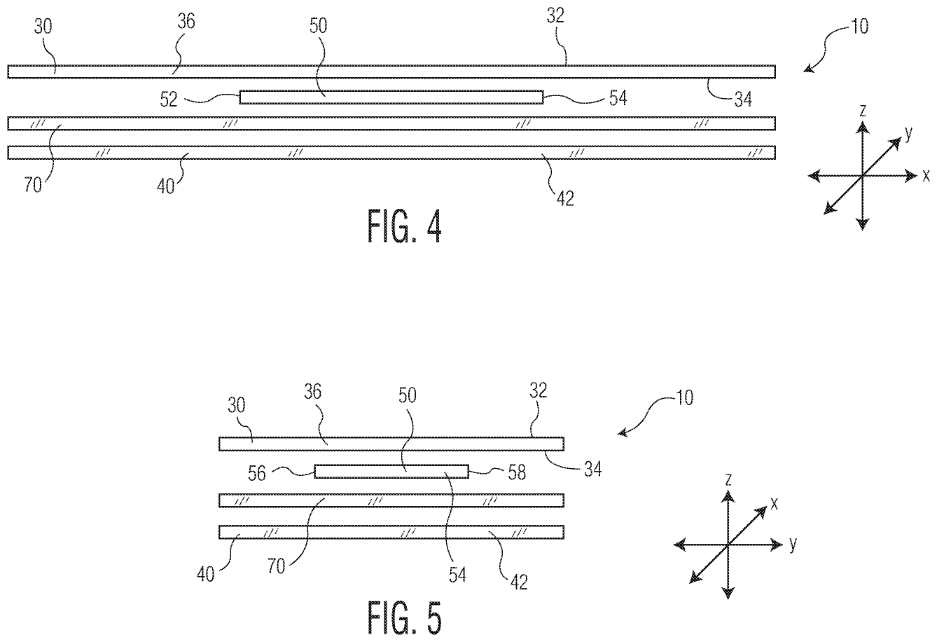

The secondary absorbent layer 70 can be incorporated into the absorbent article 10 in any suitable size and shape based upon the need of the particular absorbent article 10 in which the secondary absorbent layer 70 is being used. In various embodiments, the secondary absorbent layer 70 can extend across the entire absorbent article 10 in the longitudinal direction (X) and transverse direction (Y), such that the secondary absorbent layer 70 can have the same dimensions as the topsheet layer 30, such as, for example, illustrated in FIGS. 4, 5, and 8. In various embodiments, the secondary absorbent layer 70 can have a smaller overall length in the longitudinal direction (X) and a smaller overall width in the transverse direction (Y) than the topsheet layer 30. In various embodiments, the overall length of the secondary absorbent layer 70 can be from about 30, 40 or 50% to about 98, 99 or 100% of the overall length of the topsheet layer 30. In various embodiments, the overall width of the secondary absorbent layer 70 can be from about 10, 25 or 50% to about 98, 99 or 100% of the overall width of the topsheet layer 30.

In various embodiments, the secondary absorbent layer 70 can have an opacity of less than 30%. In various embodiments, the secondary absorbent layer 70 can have an opacity of less than 25%.

Backsheet Layer:

The backsheet layer 40 is generally liquid impermeable and is the portion of the absorbent article 10 which faces the garments of the wearer. The backsheet layer 40 can permit the passage of air or vapor out of the absorbent article 10 while still blocking the passage of liquids. Any liquid impermeable material may generally be utilized to form the backsheet layer 40. The backsheet layer 40 can be composed of a single layer or multiple layers, and these one or more layers can themselves comprise similar or different materials. Suitable backsheet material 42 that may be utilized to form a backsheet layer 40 can be a microporous polymeric film, such as a polyolefin film or polyethylene or polypropylene, nonwovens, and nonwoven laminates, and film/nonwoven laminates. The particular structure and composition of the backsheet material 42 forming the backsheet layer 40 can be selected from various known films and/or fabrics with the particular material being selected as appropriate to provide the desired level of liquid barrier, strength, abrasion resistance, tactile properties, aesthetics, and so forth. In various embodiments, a polyethylene film can be utilized that can have a thickness in the range of from about 0.2 or 0.5 mils to about 3.0 or 5.0 mils. An example of a backsheet layer 40 can be a polyethylene film such as that obtainable from Pliant Corp., Schaumburg, Ill., USA. The backsheet layer 40 can be of a single or multiple layer construction, such as of multiple film layers or laminates of film and nonwoven fibrous layers. Suitable backsheet layers 40 can be constructed from materials such as those described in U.S. Patent No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

In various embodiments, the backsheet material 42 forming the backsheet layer 40 can have an opacity of less than 30%. In various embodiments, the backsheet material 42 forming the backsheet layer 40 can have an opacity of less than 25%. As described herein, the opacity of the topsheet material 36 forming the topsheet layer 30 in combination with the size of the openings 60 can visually highlight the presence of the openings 60 within the topsheet layer 30. Additionally, the openings 60 within the topsheet layer 40 in combination with the lower opacity of the backsheet layer 40 and, in various embodiments, the lower opacity of the secondary absorbent layer 70, allow for the wearer of the absorbent article 10 to see through the openings 60 and through the lower opacity backsheet layer 40 (in comparison with the opacity of the topsheet layer 30) and, in various embodiments, through the lower opacity secondary absorbent layer 70 (in comparison with the opacity of the topsheet layer 30) to see their undergarment within which the absorbent article 10 has been placed.

An absorbent article 10, therefore, can be designed to provide discretion as the openings 60 within the topsheet layer 30 can permit the wearer's undergarment to be visible through the openings 60 thereby allowing the absorbent article 10 to blend in with the wearer's undergarment and the absorbent article 10 can be designed to indicate to the wearer that the absorbent article 10 can be breathable as the higher opacity topsheet layer 30 can define the presence of the openings 60 which allow for breathability.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. An absorbent article comprising:
a. a perimeter defined by a first transverse direction end edge and a second transverse direction end edge opposed to the first transverse direction end edge and an opposing pair of longitudinal direction side edges extending between and connecting the first transverse direction end edge and the second transverse direction end edge;
b. a topsheet layer comprising:
i. a topsheet material having an opacity of at least 40%, a body facing surface, and an absorbent core facing surface;
ii. an area defined by the perimeter of the absorbent article;
iii. a plurality of openings extending from the body facing surface of the topsheet material to the absorbent core facing surface of the topsheet material wherein each opening provides an open space within the area of the topsheet layer;
c. a backsheet layer comprising a backsheet material having an opacity less than 30%; and
d. an absorbent core positioned between the topsheet layer and the backsheet layer.

2. The absorbent article of claim 1 wherein the opacity of the topsheet material is at least 45%.

3. The absorbent article of claim 1 wherein the opacity of the backsheet material is less than 25%.

4. The absorbent article of claim 1 wherein an opacity of the absorbent core is at least the same as the opacity of the topsheet material.

5. The absorbent article of claim 1 wherein an opacity of the absorbent core is greater than the opacity of the topsheet material.

6. The absorbent article of claim 1 wherein a size dimension of the absorbent core is from 15% to 60% of a size dimension of the absorbent article.

7. The absorbent article of claim 1 wherein the area of the topsheet layer contains from 15% to 40% of open space.

8. The absorbent article of claim 1 wherein each opening of the plurality of openings has a size dimension greater than 0.3 mm².

9. The absorbent article of claim 8 wherein each opening of the plurality of openings has a size dimension greater than 0.7 mm².

10. The absorbent article of claim 1 further comprising a secondary absorbent layer.

11. The absorbent article of claim 10 wherein the secondary absorbent layer is positioned between the absorbent core and the backsheet layer.

12. The absorbent article of claim 10 wherein the secondary absorbent layer has an opacity less than 30%.

13. The absorbent article of claim 12 wherein the secondary absorbent layer has an opacity less than 25%.
